(19) United States
(12) Patent Application Publication
(10) Pub. No.: US 2006/0024815 A1
(43) Pub. Date: Feb. 2, 2006

(54) High sensitivity array-based detection system

(76) Inventors: Steve L. Allman, Knoxville, TN (US); Hannah D. Farquar, Oak Ridge, TN (US); Chung-Hsuan Chen, Knoxville, TN (US)

Correspondence Address: AKERMAN SENTERFITT, P.O. BOX 3188, WEST PALM BEACH, FL, 33402-3188, US

(21) Appl. No.: 10/903,569

(22) Filed: Jul. 30, 2004

(51) Int. Cl. C12M 1/34

(52) U.S. Cl. 435/287.2

(57) ABSTRACT

A high sensitivity array-based detection system includes a plurality of probes immobilized onto a plurality of locations provided on a surface of a patterned array, such as a microarray. At least one target selectively binds to at least one of the plurality of probes when disposed proximate thereto under appropriate conditions, such as temperature and pH. The target includes at least one bound particle. The bound particle includes a plurality of label molecules. Alternatively, the target can be immobilized to the surface of the array, such as from a sample suspected of including the target. An excitation light source provides excitation light incident to one or more locations on the patterned array. A photodetector detects emanated signals from the patterned array. The photodetector can be a commercially available digital camera.

### STATEMENT REGARDING FEDERALLY SPONSORED RESEARCH OR DEVELOPMENT

The United States Government has certain rights in this invention pursuant to contract No. 2089Q362A1 with the National Institute of Justice.

### CROSS-REFERENCE TO RELATED APPLICATIONS

Not applicable.

### FIELD OF THE INVENTION

The invention relates to methods and systems for detection of target biological materials using probes immobilized on the surface of an array.

### BACKGROUND OF THE INVENTION

Traditional methods in molecular biology generally work on a one gene per experiment basis. As a result, the throughput is very limited and a complete picture of gene function is hard to obtain. In the past several years, a new technology, commonly referred to as a DNA microarray, has attracted tremendous interests among biologists.

An array is an orderly arrangement of samples. It provides a medium for matching known and unknown DNA samples based on base-pairing rules and automating the process of identifying the unknowns. Regarding DNA microarrays, two major applications are identification of sequence (gene/gene mutation) and determination of expression level based on the abundance of corresponding mRNA or proteins. The development of microarray DNA hybridization technology is regarded as critically important for high throughput DNA analysis which is required for most large scale genomic and proteomic research.

Base-pairing (i.e., A-T and G-C for DNA; A-U and G-C for RNA) or hybridization is the underlining principle of DNA microarrays and is based on strong complementary hydrogen bonding. Two single-stranded DNA or RNA with complementary sequences can hybridize under the right conditions such as temperature, pH, and salt concentration.

The sample spot size for microarrays is typically 200 microns or less. With a microarray configuration for DNA hybridization, thousands of hybridization reactions can be pursued simultaneously on a 1 in² chip.

Protein array technology has also emerged. Protein and antibody arrays offer great promise in the application of medical diagnostics, biomarker discovery, and proteomics. Protein arrays offer a degree of versatility that is not found in DNA arrays. DNA is linear and relatively static, while protein structure is not. Adjusting the array allows identification of the protein of interest but also leads to information on cellular signaling pathways and previously unknown protein-protein interactions.

At present time, nearly all array applications, such as DNA hybridization, is detected by a laser scanner which is very expensive. The price of a commercial laser scanner for hybridization detection is typically higher than $60,000. The detection is based on laser induced fluorescence of dye molecule tagged DNA fragments. Since only one dye molecule is tagged to one DNA molecule in most cases, the detection system needs a high sensitivity detection device for photodetection. The required detection sensitivity for such systems is typically at about 10⁸ dye-tagged DNA molecules.

### SUMMARY

A high sensitivity array-based detection system includes a plurality of probes and at least one target which selectively binds to at least one of the plurality of probes when disposed proximate to the probe. One of the plurality of probes and the target are immobilized onto a plurality of locations on a surface of a patterned array while the other of the plurality of probes and the target include at least one bound particle, the bound particle includes a plurality of label molecules.

The particle can be bound to the non-immobilized one of the plurality of probes and the target before or after completion of the selective binding (e.g. DNA hybridization). An excitation light source provides excitation light incident to one or more locations on the patterned array. Due to the high detection sensitivity provided by systems according to the invention, it is no longer necessary to have an intense laser source for excitation. A modest light source, such as an LED, laser diode array based device or a discharge UV light device can be used instead of a conventional laser. A photodetector detects emanated signals from the patterned array. The photodetector can comprise a CCD-based digital camera.

The label can be a fluorescent dye and the emanated signals can be fluorescent signals. In this embodiment, the system preferably includes an optical filter in an optical path between the array and the detector for rejecting the excitation light. The label can also be a non-fluorescent label and the emanated signals can be scattered signals. The system can include a mirror for reflecting the emanated signals disposed on a side of the array opposite a side radiated by the excitation source.

In one embodiment, the plurality of label molecules provide at least two different wavelengths of emanated signals. The plurality of probes can include oligonucleotide and/or protein probes.

A method of detecting target biomolecules using arrays includes the steps of providing an array including a plurality of probes and at least one target which selectively binds to at least one of the plurality of probes when disposed proximate to the probe. The plurality of probes or the target are immobilized onto a plurality of locations on a surface of a patterned array while the other of the plurality of probes and the target includes at least one bound particle, the bound particle comprising a plurality of label molecules. The array is exposed to a sample suspected of including the target when the plurality of probes are immobilized or the plurality of probes when the target is immobilized. One or more locations on the patterned array are irradiated with excitation light. The presence or absence of the target are then determined based on the detection of emanated signals from the patterned array.

The method can include the step of forming an image from the emanated signals, such as using a CCD-based digital camera. The plurality of probes can comprise oligonucleotide probes or protein probes. The particle can comprise a nanoparticle or microparticle, the particle including at least 10⁵ labels. In one embodiment, the plurality of label molecules provide at least two different wavelengths of emanated signals. Such an arrangement enables multiplexed detection.

The plurality of labels can be bound to the non-immobilized one of the plurality of probes and the target after the selective binding takes place. The labels can comprise magnetic or metallic comprising particles. In another embodiment, both the plurality of probes and the target include bound particles comprising a plurality of label molecules. In this embodiment, the method can comprise fluorescent resonant energy transfer (FRET).

### BRIEF DESCRIPTION OF THE DRAWINGS

A fuller understanding of the present invention and the features and benefits thereof will be accomplished upon review of the following detailed description together with the accompanying drawings, in which:

FIG. 1 is a schematic for an exemplary high sensitivity array-based detection system according to the invention.

FIG. 2 shows image results obtained based on fluorescent light measurement using system 100 shown in FIG. 1 for target concentrations of 1 fmole, 10 fmole and 100 finole.

FIG. 3 shows image results obtained based on light scattering measurement using a PC scanner for target concentrations of 1 fmole, 10 fmole and 100 fmole.

### DETAILED DESCRIPTION

Before proceeding further with a description of the specific embodiments of the present invention, a number of terms will first be defined.

A “target” is a molecule that has an affinity for a given probe (defined below). Targets may be naturally occurring or synthetic. The target may be, for example, proteins including antibodies, cell membrane receptors, drugs, polynucleotides, nucleic acids, peptides, cofactors, sugars, cells, and organelles.

A “probe” is a molecule that is recognized by a particular target. The probe may be one of the molecules defined above under “target”.

A “polynucleotide” is a compound or composition that is a polymeric nucleotide or nucleic acid polymer. The polynucleotide may be a natural compound or a synthetic compound. In the context of an assay, the polynucleotide is generally referred to as a polynucleotide analyte. The polynucleotides include nucleic acids, and fragments thereof, from any source in purified or unpurified form including DNA and RNA, including tRNA, mRNA, rRNA, mitochondrial DNA and RNA, chloroplast DNA and RNA, DNA/RNA hybrids, or mixtures thereof, genes, chromosomes, plasmids. In addition, the genomes of biological material such as microorganisms, such as bacteria, yeasts, viruses, viroids, molds, fungi, plants, animals, humans, and the like. The polynucleotide can be only a minor fraction of a complex mixture such as a biological sample. Also included are genes, oncogenes, cDNA, and the like.

The polynucleotide can be obtained from various biological materials by well known procedures. The polynucleotide, where appropriate, may be cleaved to obtain a fragment that contains a target nucleotide sequence, for example, by shearing or by treatment using a site specific chemical cleavage method.

A “target nucleotide sequence” is a sequence of nucleotides to be identified, usually existing within a portion or all of a polynucleotide, usually a polynucleotide analyte. The identity of the target nucleotide sequence generally is known to an extent sufficient to allow preparation of various sequences hybridizable with the target nucleotide sequence and of oligonucleotides, such as probes and primers, and other molecules necessary for conducting methods in accordance with the present invention, an amplification of the target polynucleotide, and so forth.

An “oligonucleotide” is a polynucleotide, usually single stranded, usually a synthetic polynucleotide but may be a naturally occurring polynucleotide. The oligonucleotide(s) are usually comprised of a sequence of at least 5 nucleotides, generally 10 to 100 nucleotides, and preferably 10 to 30 nucleotides.

An “oligonucleotide probe” is an oligonucleotide employed to bind to a portion of a polynucleotide such as another oligonucleotide or a target nucleotide sequence. The design and preparation of the oligonucleotide probes are generally dependent upon the sensitivity and specificity required, the sequence of the target polynucleotide and, in certain cases, the biological significance of certain portions of the target polynucleotide sequence.

A “nucleotide” is a base-sugar-phosphate combination that is the monomeric unit of nucleic acid polymers, i.e., DNA and RNA. The nucleotide may be natural or synthetic. The term “nucleotide” as used herein includes modified nucleotides that contain a modified base, sugar or phosphate group.

A “label” is a moiety that is capable of being activated usually by light and of producing a detectable light signal such as in, for example, fluorescence, and phosphorescence. The label can be a fluorescent group, preferably having a large Stokes shift, such as fluorescein, rhodamine, hexachlorofluorescein, tetramethylrhodamine, dichlorofluorescein, indocarbocyanine dyes, ethidium bromide, chelated lanthanides, phycoerythrin, GFP, and the like. Other types of labels include, for example, other dye particles, such as those involving fluorescence resonance energy transfer, and so forth. Usually, the label is part of a target nucleotide sequence or an oligonucleotide probe, either being conjugated thereto or otherwise bound thereto or associated therewith.

An “array” is an arrangement of a plurality of features or objects in on a substrate (e.g. chip) surface in which each object occupies a separate predetermined spatial position. Typically, the objects have a predetermined arrangement in the x-axis and the y-axis, thus forming rows and columns. For arrays, the surface of the substrate can comprise from about 10¹ (focus arrays) to about 10⁸ different features such as attached polynucleotides, each in an area of from several square microns to about 1000×1000 micron. However, for focus arrays, spot areas are generally greater than this value.

Now returning to the invention, a high sensitivity array-based detection system includes a plurality of probes immobilized onto a plurality of locations provided on a surface of a patterned array, such as a microarray. At least one target selectively binds to at least one of the plurality of probes when disposed proximate thereto under appropriate conditions, such as temperature and pH. The target includes at least one bound particle. The bound particle includes a plurality of label molecules. An excitation light source provides excitation light incident to one or more locations on the patterned array. A photodetector detects emanated signals from the patterned array, such as a commercially available CCD-based digital camera.

Alternatively, the target can be immobilized to the array substrate, such as from a sample suspected of including the target. In this embodiment, the particles having a plurality of labels are bound to the probes and the detection is based on the detection of probes. Otherwise, the system and detection method is same as with immobilized probes.

Both the probe and the target can be labeled according to the invention. In this embodiment, the particle, such as a polymeric bead, has a plurality of first labels and is attached to the slide surface of the array, and the probe is attached to the bead. The target, with a particle having a plurality of second labels, selectively binds to the probe. If a matched duplex is attained, Fluorescence Resonance Energy Transfer (FRET) occurs through reaction between the particle on the probe and the particle on the target.

The bound particle is generally a nano or micro scale particle. Each particle contains at least 10⁵ and generally more than 10⁸ label molecules. Particles can be based on polymeric materials. When metallic (e.g. gold, silver), or magnetic (γ iron oxide, cobalt) particles are used, measurements can be obtained based on the scattering light of particles. Since as low as one target molecule can be attached onto a single label comprising particle, the detection sensitivity can be many orders of magnitude higher than conventional single label molecule tagging. Although it is currently difficult to put fluorescent molecules inside of a metal particle or a magnetic particle, a plastic particle can include both magnetic materials and label molecules so that magnetic and/or fluorescent properties can be used for detection.

Due to the high detection sensitivity provided by systems according to the invention, it is no longer necessary to have an intense laser source for excitation. A modest light source, such as an LED or laser diode array based device or a discharge UV light device can be used instead of a conventional laser.

Regarding photodetection, an imaging detector or a non-imaging detector can be used. In a preferred embodiment, the detector is an imaging detector. For example, the imaging detector can comprise a CCD device used in a typical digital camera. With this system arrangement, the cost for an array detection system can be generally reduced to less than $2,000.

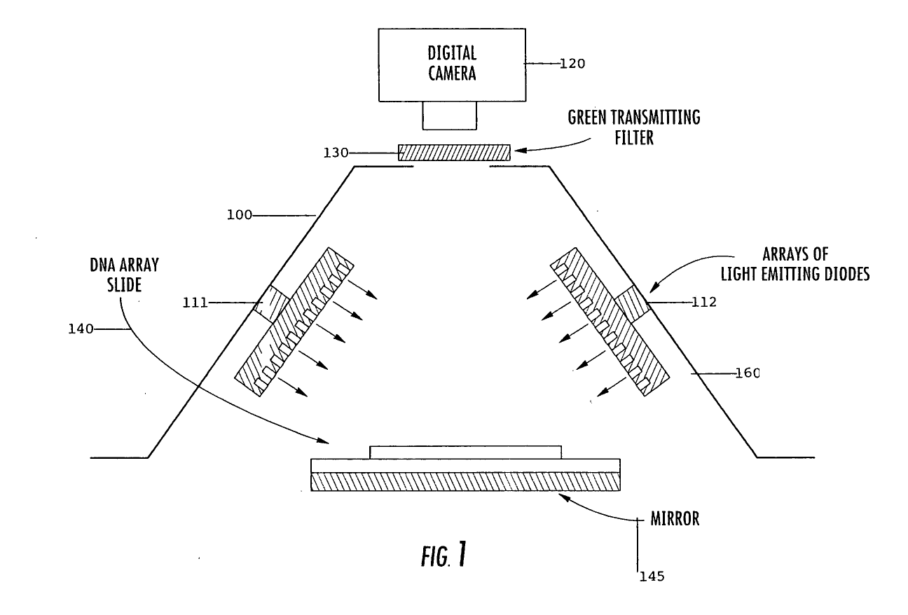

A schematic for an exemplary high sensitivity detection system according to the invention is shown in FIG. 1. System 100 includes two (2) arrays of light emitting diodes 111 and 112 which shine light onto the surface of a DNA slide array 140. The photodetector shown is a digital camera 120 which includes a charge-coupled device (CCD) array.

Although generally described relative to DNA hybridization-based systems, the invention is in no way limited to such systems. For example, the invention can be applied to protein antibody-antigen interactions, aptamer microarray and protein interactions, or other binding-based interactions of interest.

Although a CCD-array based digital camera 120 is shown in FIG. 1, the photodetector may also be based on photomultiplier tubes, photodiodes, phototransistors, avalanche diodes, and so forth. An array detector may be used to measure individually the signal from each light source. At least one detector element is used to measure the signal from each light source in an array. However, more than one detector may be employed to over-sample the targets to permit discrimination against non-uniformities.

The LED arrays 111 and 112 and the slide array 140 are shown contained inside a light-tight box 160 to shield ambient room light. An aperture in box 160 is covered by optical filter 130 which passes the a fluorescent signal emanated by the labels while rejecting light from LEDs 111 and 112 scattered by the array 140.

Array 140 can be a commercial microarray DNA hybridization chip for DNA hybridization reactions. In this case, the target DNA or other target is labeled with a nano or micro particle having a large number of label molecules, such as 10⁸, or more. For example, the particle can be a polystyrene bead impregnated with a fluorescent dye such as Dragon Green provided by Bang Labs. Inc. (Fishers, Ind.).

Target detection can be based on the measurement of either scattered light or the fluorescence (or phosphorescence) from the particles on the array sites. A mirror 145 is shown placed behind of the array 140 to reflect both scattering light and fluorescent light to enhance the detection efficiency by the digital camera 120. Mirror 145 may not be required for a digital camera detection.

Although the excitation light source shown in FIG. 1 utilizes LED arrays 111 and 112, the light source can be a single light source. Examples of other suitable light sources include, by way of illustration and not limitation, laser diodes, laser such as argon, helium, neon, dye titanium sapphire, VCELs or other intense discharge light sources.

Typically, the excitation light source illuminates the sample with an excitation wavelength that is within the visible spectrum, but other wavelengths such as near ultraviolet or near infrared spectrum may be used depending on the nature of labels employed, the nature of the sample to be analyzed, and the number of different dyes used in the system. In some instances, excitation is with electromagnetic radiation having a wavelength at or near the absorption maximum of the dye.

For fluorescence measurements, a light source with short wavelength (i.e. UV or blue light region) is highly preferred. However, when commercial UV diode lasers become available at a modest price, the detection sensitivity can be further improved without significantly increasing the cost of the system.

When embodied as an array of excitation light sources, the light source can comprises 2 to about 100,000, usually, about 10 to about 1,000 light sources. Each of the light sources in the plurality of light sources is usually the same but need not be. The array of light sources has the light sources arranged in a predetermined pattern such as a certain number of light sources in rows and columns, for example, a 50×50 array, or one-dimensional such as a 1000×1 array. The nature of the arrangement in a plurality of light sources is dependent on the ease and scalability of manufacturing processes for the light source array.

For scattering detection, both fluorescent or non-fluorescent particles can be used. As noted above, for scattering detection, magnetic and gold nanoparticles can also be used. Optical filter 130 is not required for non-fluorescent scattering light detection. In generally, the scattering light signal is stronger than the fluorescent light signal. However, when scattering is used, dust particles on the array 140 can also serve as a source for light scattering to produce false positives.

For fluorescent measurements, filter 130 blocks the shorter wavelength scattering light to reduce background noise. Regarding fluorescent measurements, dust particles on the chip should not give rise to detected interference.

Label particle tagging can be processed after DNA hybridization or other binding event is completed. With this approach, DNA immobilization and hybridization or other probe-target binding can completely follow commercial protocol. For example, the target DNA can be labeled with biotin so that dye particles can be attached through the biotin-streptavidin reaction. The surface of dye particles can be coated with streptavidin molecules. A UV cross linking process can be used after the hybridization before the tagging of dye particles onto the hybridized duplex through biotin-streptavidin reaction.

If particle labeling is performed after completion of the binding step, the use of different labels such as multiple color dye will provide no advantage. However, if different type of targets are labeled with particles having different dyes prior to binding, the advantages of multiplexing will be provided. For example, if person A's DNA is labeled with a red color emanating particle and person B's DNA is labeled with a green color emanating particle, hybridization studies can be pursued for both people at the same time.

### EXAMPLES

The present invention is further illustrated by the following specific Examples, which should not be construed as limiting the scope or content of the invention in any way.

An experiment was run utilizing system 100 shown in FIG. 1 for hybridization detection. A commercial DNA chip (Codelink Amersham, Piscatawny, N.J.) was used to immobilize probe DNA and the subsequent hybridization process. Hybridizations were performed on CodeLink Activated Slides.

Probe oligonucleotides (5′-amino modified) were spotted onto the surface according to the manufacturer recommendations. Briefly, a solution of probe oligonucleotide in 150 mM Na₂PO₄ buffer (pH 8.5) was manually spotted onto the surface of array 140 and allowed to react overnight in a 75% humidity chamber. The spots were approximately 1 mm in size when a volume of 0.5 μL was used. The slides were then incubated at 50° C. for 30 min in a blocking solution (0.1 M Tris, 50 mM ethanolamine, pH 9.0) to block un-reacted sites on the slides. The slides were then washed with water and incubated at 50° C. for 30 min in 4×SSC, 0.1% SDS followed by a water wash and dried with in a centrifuge at 800 rpm. Hybridizations were carried out in a Secure Seal Hybridization Chamber (Grace Bio Labs) in ˜85 μL of target in 4×SSC, 0.1% SDS for 2 h. Hybridizations were performed at 50° C. for the following sequence:

| | |
|---|---|
| Probe: | 5′-NH2-C12 spacer- GAATAATATTTTTCCAAGAAAGA |
| | ACTAC- -3′ |
| Target: | 5′-biotin- TGGCTTGTAGTTCTTTCTTGGAAAAATA |
| | TTA- 3′ |

The target sequence was labeled with a Dragon Green microsphere (Bangs Laboratories, Inc.) as per the protocol of the manufacturer. Briefly, the storage buffer was removed from an aliquot of microspheres (100 μL, 1 mg). For each step the microspheres were separated from the buffer by centrifuging at 14,000 rpm for 15 min. The microspheres were then washed with TTL buffer (100 mM Tris-HCI pH 8.0, 0.1% Tween 20, 1 M LiCl) twice. After addition of the buffer the microspheres were aspirated with a disposable pipette and sonicated for 1 hour to fully suspend the microspheres. The microspheres were then suspended in TTL buffer (20 μL) to which the target oligonucleotide was added.

After reacting for 15 min at room temperature with gentle shaking, 0.15 N NaOH was added. The solution was separated from the microspheres and the microspheres attached to the oligonucleotide were washed twice with TT buffer (250 mM Tris-HCl pH 8.0, 0.1% Tween 20). The microspheres attached to the oligonucleotide were then resuspended in TTE buffer (250 mM Tris-HCl pH 8.0, 0.1% Tween 20, 20 mM Na₂ EDTA pH 8.0) and heated to 80° C. for 10 min. The microspheres were then separated from the TTE buffer and resuspended in hybridization buffer.

After hybridization, wash conditions consisted of 2×SSC, 0.1% SDS for 5 min at 50° C. twice, 0.2×SSC for 1 min at room temperature, 0.1×SSC for 1 min at room temperature, and a final water wash at room temperature. The slides were then dried by centrifugation at 800 rpm before visualization by digital camera 120.

In order to observe the fluorescence from hybridization spots using digital camera 120, the two clusters of 40 blue light emitting diodes (LEDs) 111 and 112 were positioned at approximately 45-degree angles with respect to the surface DNA slide array 140 containing the spots of hybridized DNA attached to fluorescent beads. The glass slide was mounted on a holder in such a way that it is held in place by its edges, allowing the light from the LEDs to pass through the optically transparent glass slide on array 140. The LEDs and the glass slide were contained inside light-tight box 160 to shield ambient room light, with the glass slide holder positioned well above the bottom of the box. Positioning the slide away from the bottom allows almost all of the LED light to pass through the slide and not be reflected upwards, toward the camera 120. A small opening in the top of the box was covered band pass filter 130 selected to transmit the fluorescent green light emitted by the beads, while blocking the blue excitation light emitted by the LEDs.

Digital camera 120 was positioned just above the filter 130, and takes a picture of the slide. Because of the geometric arrangement of the lights 111 and 112, slide array 140, and camera 120, the fluorescence emitted by the beads is visible with minimal background noise from scattered and reflected illumination. A typical picture requires a several second exposure, because the amount of light emitted from the fluorescent beads is quite small. Afterward, the digital images can be viewed on a computer, or printed out on a color printer, in order to determine which spots on the slide have bound targets and are thus visible due to the presence of fluorescent beads.

As noted above, in addition to fluorescent measurement, hybridization can also be observed by scattered light directly. With scattering light measurements, the excitation light source can generally emit in a broader range of wavelengths as compared to fluorescence. In this embodiment, the particles for tagging are no longer limited to fluorescent nanoparticles, such as Dragon Green. For example, as noted above gold and magnetic nano and microparticles can also be used.

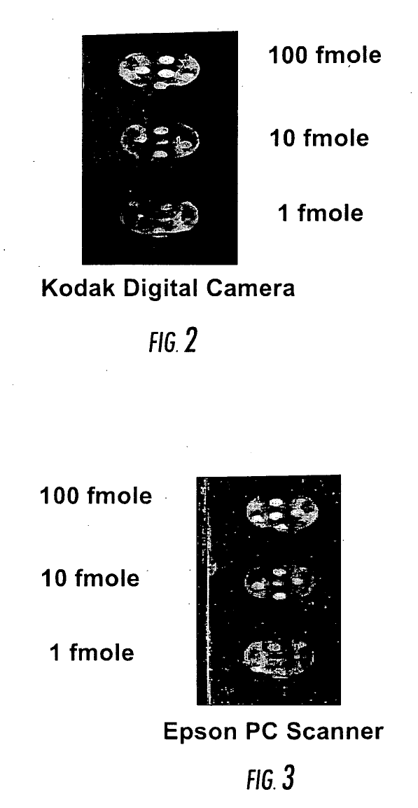

Results obtained based on fluorescent light measurement using system 100 including a digital camera is shown in FIG. 2 for target quantities of 1 fmole, 10 fmole and 100 fmole. Test of similar samples with light scattering detection is shown in FIG. 3 using a PC scanner instead of a digital camera.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

We claim:

1. An array-based detection system, comprising:
a plurality of probes;
at least one target which selectively binds to at least one of said plurality of probes when disposed proximate thereto, wherein one of said plurality of probes and said target are immobilized onto a plurality of locations on a surface of a patterned array while the other of said plurality of probes and said target includes at least one bound particle, said bound particle comprising a plurality of label molecules;
an excitation light source providing excitation light incident to one or more locations on said patterned array, and
a photodetector for detecting emanated signals from said patterned array.

2. The system of claim 1, wherein said label is a fluorescent dye and said emanated signals are fluorescent signals, further comprising an optical filter in a path between said array and said detector for rejecting said excitation light.

3. The system of claim 1, wherein said label is a non-fluorescent label and said emanated signals are scattered signals.

4. The system of claim 1, further comprising a mirror for reflected said emanated signals disposed on a side of said array opposite a side radiated by said excitation source.

5. The system of claim 1, wherein said excitation light source comprises an LED array or a laser diode array.

6. The system of claim 1, wherein said photodetector comprises a photodiode, an avalanche photodiode, a CCD, a CMOS sensor, or an array thereof.

7. The system of claim 1, wherein said photodetector comprises a CCD-based digital camera.

8. The system of claim 1, wherein said plurality of label molecules provide at least two different wavelengths of said emanated signals.

9. The system of claim 1, wherein at least one of said plurality of probes is a protein.

10. A method of detecting target biomolecules using arrays, comprising the steps of:
providing an array including a plurality of probes, at least one target which selectively binds to at least one of said plurality of probes when disposed proximate to said at least one probe, wherein one of said plurality of probes and said target are immobilized onto a plurality of locations on a surface of a patterned array while the other of said plurality of probes and said target includes at least one bound particle, said bound particle comprising a plurality of label molecules;
exposing said array to a sample suspected of including said target when said plurality of probes are immobilized or said plurality of probes when said target is immobilized,
irradiating said one or more locations on said patterned array with excitation light, and
determining a presence or absence of said target based on detection of emanated signals from said patterned array.

11. The method of claim 10, further comprising the step of forming an image from said emanated signals.

12. The method of claim 11, wherein said image is formed by a CCD-based digital camera.

13. The method of claim 10, wherein said plurality of probes comprise oligonucleotide probes.

14. The method of claim 10, wherein said plurality of probes comprise protein probes.

15. The method of claim 10, wherein said particle comprises a nanoparticle or microparticle, said particle including at least 10⁵ labels.

16. The method of claim 10, wherein said plurality of label molecules provide at least two different wavelengths of said emanated signals, said method comprising multiplexed detection.

17. The method of claim 10, wherein said plurality of labels are bound to said target or said plurality of probes after said selective binding.

18. The method of claim 10, wherein said labels comprise magnetic or metallic comprising particles.

19. The method of claim 10, wherein both said plurality of probes and said target include said bound particles comprising a plurality of label molecules.

20. The method of claim 19, wherein said method comprises fluorescent resonant energy transfer (FRET).


SEQUENCE LISTING (2 sequences)

SEQ ID NO: 1 - DNA, 27 bp, Artificial Sequence
  Probe
aataatatttttccaagaaagaactac

SEQ ID NO: 2 - DNA, 31 bp, Artificial Sequence
  Target sequence
tggcttgtagttctttcttggaaaaatatta